(19) United States
(12) Patent Application Publication
(10) Pub. No.: US 2003/0010460 A1
(43) Pub. Date: Jan. 16, 2003

(54) Method and device for monitoring the state of felt or of a screen

(76) Inventors: Joachim Grabscheid, Gerstetten (DE); Ulrich Begemann, Heidenheim (DE); Oswald Satzger, Glengen (DE); Wolfgang Mayer, Heidenheim (DE); Ralf Rziha, Gerstetten (DE); Wolfgang Bamberger, Heidenheim (DE); Georg Kleiser, Heidenheim (DE); Thomas Ruhl, Wernau (DE)

Correspondence Address: Todd T. Taylor, TAYLOR & AUST, P.C., 142 S. Main St., P.O. Box 560, Avilla, IN, 46710, US

(21) Appl. No.: 10/194,872

(22) Filed: Jul. 12, 2002

(30) Foreign Application Priority Data
Jul. 13, 2001 (DE) 101 34 234
Jul. 30, 2001 (DE) 101 37 094

(51) Int. Cl. D21F 1/32; D21F 7/06; G01N 33/34; G01N 15/00

(52) U.S. Cl. 162/199; 162/198; 162/263; 162/272; 73/38

(57) ABSTRACT

In a method of monitoring the state of a felt serving for the dewatering of a fiber web, in particular of a paper web or of a cardboard web, the felt topography is measured and/or an image of the felt surface having definition in depth is produced by mean of an optical measuring device and the result of the measurement obtained is compared with previously stored error patterns by means of an evaluation and/or control unit. Corresponding felt conditioning measures are then initiated, preferably automatically, in dependence on the result of the comparison. A corresponding felt monitoring device is also described. In a further method of monitoring the state of a felt or screen serving the dewatering of a fiber web, in particular of a paper web or a cardboard web, specific felt and/or screen properties are determined permanently or cyclically at intervals and are filed under a pre-settable felt and/or screen name in a historical database. By means of an evaluation and/or control unit, the relevant data are graphically recorded, in particular relative to the time of the felt and/or screen change, the obtained property development over time is compared with a felt and/or screen property development already previously determined or recorded and a signaling is initiated and/or at least one counter measure is automatically triggered if the obtained property development over time differs from the felt and/or screen property development over time already previously determined or recorded by a pre-settable measure or lies outside a pre-settable range. A corresponding monitoring device is also described.

### BACKGROUND OF THE INVENTION

1. Field of the Invention

The invention relates to a method and to a device for monitoring the state of a felt or of a screen serving for the dewatering of a fiber web, in particular of a paper web or of a cardboard web.

2. Description of the Related Art

Rotating felts or screens are used in particular for dewatering in paper making machines. A worsening of the dewatering performance and of the evenness of a respective felt and/or screen has a corresponding negative effect on the paper quality and/or on the production performance. Felts and screens have previously been monitored with respect to their effect, such as in particular the dewatering performance and/or the like. If now a worsening of the dewatering performance and of the evenness of the felt and/or screen is found, this already has a negative effect on the paper quality and/or the production performance.

### SUMMARY OF THE INVENTION

It is the aim of the invention to provide an improved method and an improved device of the kind initially mentioned in which the risk of an impairment of the product quality and/or of the production performance is reduced to a minimum.

In accordance with a first aspect of the invention, this object is satisfied by a method of monitoring the state of a felt which serves the dewatering of a fiber web, in particular of a paper web or of a cardboard web, and in which the felt topology is measured or an image of the felt surface having definition in depth is produced by means of an optical measuring device and the result of the measurement obtained is compared with previously stored error patterns by means of an evaluation and/or control unit, and in which corresponding felt conditioning measures are initiated, preferably automatically, in dependence on the result of the comparison.

It is thus possible in a simple and reliable manner to recognize a worsened felt state so early that measures can still be taken in time to counter an impairment of the manufacturing process and thus an impairment of the product quality and/or of the production performance. The stored error patterns can in particular be typical error patterns that occur in practice.

A plan image and/or an oblique image of the felt surface is preferably produced by means of the optical measuring device.

The image produced can be recorded and/or stored.

The image produced by means of the optical measuring device can remain stored for reuse, for example as an error pattern, or only be temporarily stored and be deleted or overwritten again after a comparison has been made with the stored error patterns.

The felt is advantageously monitored continuously or periodically by means of the optical measuring device.

The felt monitoring can in particular take place online.

An optical measuring device can expediently be used with at least one camera such as, in particular, a full-frame camera.

With the preferred practical embodiment of the method in accordance with the invention, flashlight of such a high energy is used that an image having definition in depth is in particular also obtained by means of the camera at higher felt speeds.

It is also of advantage if the optical measuring device is guided in a traversing manner over the width of the felt.

The camera and the lighting can be used such that they stand at a specific angle to one another.

In the case of a sectional error recognition, in particular sectional felt conditioning measures can also be initiated.

The felt conditioning measures initiated in the case of an error detection can include, for example, the production of a tube suction vacuum, the selection of spray tube nozzles and/or the like.

The device relating to this first aspect of the invention for the monitoring of the state of a felt serving for the dewatering of a fiber web, in particular of a paper web or of a cardboard web, correspondingly includes at least one optical measuring device for the measuring of the felt topography or for the production of an imaging of the felt surface having definition in depth as well as an evaluation and/or control unit to compare the result of the measurement obtained with previously stored error patterns and to initiate corresponding felt conditioning measures, preferably automatically, in dependence on the result of the measurement.

Preferred embodiments of this device relating to the first aspect of the invention are set forth in the dependent claims 14 to 24.

In accordance with a second aspect of the invention, the initially set forth object is satisfied in accordance with the invention by a method of monitoring the state of a felt or screen serving for the dewatering of a fiber web, in particular of a paper web or of a cardboard web, in which specific felt properties and/or screen properties are determined permanently or cyclically at intervals and are filed under a pre-settable felt name or screen name in a historical database, and in which, by means of an evaluation and/or control unit, the relevant data are graphically recorded, in particular relative to the time of the felt change and/or screen change, the obtained property development over time is compared with at least one felt and/or screen property development over time which was already determined or recorded previously, and a signaling is initiated and/or at least one counter measure is automatically triggered if the obtained property development over time differs from the felt and/or screen development over time already previously determined or recorded or lies outside a pre-settable range.

In this connection, at least one counter measure can in particular also be triggered sectionally over the felt width and/or screen width. For example, the development of the relevant data over time can be recorded. Alternatively, or additionally, a processing and/or monitoring and/or controlling of sectional qualities of felt or screen properties, a sectional monitoring of the felt and/or screen properties and/or a monitoring or recognition of specific limit values and error patterns can be provided for the selection and initiating of specific counter measures, in each case over time and/or sectionally over the machine width. In this connection, a characteristic field control is in particular also possible. Generally, not only a signaling, but also an automated triggering of counter measures is possible, also sectionally over the width of the felt and/or screen, e.g.:

change in spray tube pressures (for example also sectionally);

change in tube suction vacuums (for example also sectionally);

further operating parameters which influence the measured properties and put the values in the pre-determined characteristic field again (feedback loop).

A worsening of the respective felt and/or screen state can thus be determined at an early time so that, optionally, measures can still be taken in good time which counteract an impairment of the manufacturing process and thus a worsening of the paper quality and/or of the production performance. Now an optimum felt and/or screen management and an optimum trend monitoring of the relevant properties is possible.

The felt and/or screen properties determined and/or recorded can in particular be put into relationship with a previously determined or recorded mean property trend value and/or compared with this.

The felt and/or screen properties can be determined online or offline.

For example, the occurring felt and/or screen vibrations, the felt and/or screen permeabilities, the water content of the felt and/or screen, the felt and/or screen contamination, the surface finish of the felt and/or screen and/or the like can be determined as the felt/or screen properties. In this connection, in particular the sectional qualities of the individual properties can also be determined.

The device relating to this second aspect of the invention for monitoring the state of a felt or screen serving for the dewatering of a fiber web, in particular of a paper web or of a cardboard web, in which specific felt and/or screen properties are determined correspondingly permanently or cyclically at intervals and are filed under a pre-settable felt and/or screen name in a historical database, correspondingly includes an evaluation and/or control unit for the graphical recording of the related data, in particular relative to the time of the felt and/or screen change, for the comparison of the obtained property development over time with at least one felt and/or screen property development over time already previously determined or recorded and for the signaling and/or automatic triggering of at least one counter measure in the event of a deviation of the obtained property development over time from the felt and/or screen property development over time already previously determined by a pre-settable measure or in the event of a property development over time lying outside a pre-settable range.

Preferred embodiments of this monitoring device relating to the second aspect of the invention are set forth in the dependent claims 41 to 54.

### BRIEF DESCRIPTION OF THE DRAWINGS

The invention will be described in more detail in the following with reference to embodiments and to the drawing which shows, in a purely schematic representation, an embodiment relating to a first aspect of the invention of a device for the monitoring of the state of a rotating felt serving for the dewatering of a fiber web, in particular of a paper web or of a cardboard web.

### DETAILED DESCRIPTION OF THE INVENTION

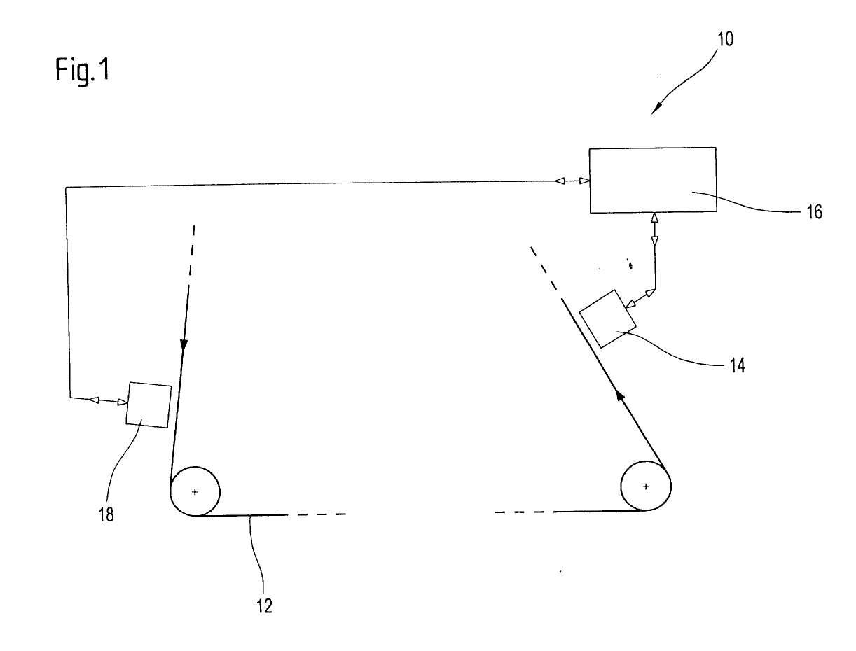

The reference numeral 10 describes a device 10 for the monitoring of the state of a rotating felt 12 serving for the dewatering of a fiber web, in particular of a paper web or of a cardboard web. The felt can be guided in a variety of manners and adopt any desired development which can in particular also deviate from the purely exemplary development shown in FIG. 1.

The monitoring device 10 includes at least one optical measuring device 14 for the measuring of the felt topography and/or for the production of an image of the felt surface having definition in depth.

This optical measuring device 14 is connected to an evaluation and/or control unit of the device 10 which can be in connection with the means 18 serving the felt conditioning. The arrangement resulting from FIG. 1 of the optical measuring device 14 and of the felt conditioning means 18 is purely exemplary and correspondingly selectable depending on the respective circumstances.

The obtained result of the measurement is compared with previously stored error patterns in the evaluation and/or control unit 16. Corresponding felt conditioning means are then preferably automatically initiated in dependence on the result of the comparison. For this purpose, the felt conditioning means 18 can, for example, be correspondingly selected via the evaluation and/or control unit 16.

A plan image and/or an oblique image of the felt surface can, for example, be produced by means of the optical measuring device 14.

The image produced by means of the optical measuring device 14 can be recorded and/or stored. In particular electronic recording means can also be used. The image produced can be stored for a longer time and be stored, for example, as an error pattern or it can only be stored temporarily and be deleted or overwritten again after a comparison has been made with the stored error patterns.

The felt 12 can be monitored continuously or periodically by means of the optical measuring device 14. In this connection, an online monitoring is also possible.

The optical measuring device 14 can include, for example, at least one camera such as in particular a full frame camera.

Expediently, a flashlight of such high energy is used that an image having definition in depth is still also obtained by means of the camera in particular with higher felt speeds The optical measuring device 14 and/or the camera can be guided in a traversing manner over the felt width.

The camera and the lighting can in particular be used such that they stand at a specific angle to one another.

In the event of a sectional error recognition, sectional felt conditioning measures can also expediently be initiated.

The felt conditioning measures initiated via the means 18 in the event of an error recognition can include, for example, the production of a tube suction vacuum, the selection of spray tube nozzles and/or the like.

A worsened felt state can be discovered so early with the method set forth and with the corresponding device 10 that corresponding measures can still be taken to counteract an impairment of the manufacturing process and thus of the paper quality and/or of the production performance.

FIG. 2 of the drawing shows, in a purely schematic representation, a device 20 in accordance with the second aspect of the invention for the monitoring of the state of a felt or screen 22 serving for the dewatering of a fiber web, in particular of a paper web or of a cardboard web.

The monitoring device 20 includes at least one corresponding measuring apparatus 24 by means of which specific felt and/or screen properties are determined permanently or cyclically at intervals. The measuring apparatus can in particular include at least one traversing unit. It further includes an evaluation and/or control unit 26.

The felt and/or screen properties determined are filed under a pre-settable felt and/or screen name in a historical database, which can be done by means of the evaluation and/or control unit 26.

The development of the relevant data over time is graphically recorded, in particular relative to the time of the felt and/or screen change, by means of the evaluation and/or control unit 26 including a corresponding calculating unit. Moreover, the obtained property development over time is compared with at least one felt and/or screen property development over time already previously determined or recorded by means of this evaluation and/or control unit and a signaling initiated if the obtained property development over time differs by a pre-settable measure from the felt and/or screen property development already previously determined or recorded or lies outside a pre-settable range.

The felt and/or screen properties determined and/or recorded can in particular be put into relationship with a mean property trend value already previously determined or be compared with this.

The felt and/or screen properties can be determined online or offline.

In particular the occurring felt and/or screen vibrations, the felt and/or screen permeabilities, the water content of the felt and/or screen, the felt and/or screen contamination, the surface finish of the felt and/or screen 22 and/or the like can be determined as the felt and/or screen properties.

A monitoring of the following parameters is, for example, also possible:

felt and/or screen thickness

section quality of the properties

recognition of sectional deviations from the pre-set characteristic field

recognition of section patterns (long wave/short wave).

Optionally, felt conditioning measures or the like can, for example, again be initiated via means 28.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A method of monitoring the state of a felt (12) serving for the dewatering of a fiber web, in particular of a paper web or of a cardboard web, in which the felt topography is measured and/or an image of the felt surface having definition in depth is produced by means of an optical measuring device (14) and the obtained result of the measurement is compared with previously stored error patterns by means of an evaluation and/or control unit (16), and in which corresponding felt conditioning measures are initiated, preferably automatically, in dependence on the result of the comparison.

2. A method in accordance with claim 2, characterized in that a plan image and/or an oblique image of the felt surface is produced by means of the optical measuring device (14).

3. A method in accordance with claim 1, characterized in that the image produced by means of the optical measuring device (14) is recorded and/or stored.

4. A method in accordance with claim 2, characterized in that the image produced by means of the optical measuring device (14) is recorded and/or stored.

5. A method in accordance with claim 3, characterized in that the image produced by means of the optical measuring device (14) is only temporarily stored and is deleted or overwritten again after a comparison has been made with the stored error patterns.

6. A method in accordance claim 1, characterized in that the felt (12) is monitored continuously or periodically by means of the optical measuring device (14).

7. A method in accordance with claim 1, characterized in that the felt monitoring takes place online.

8. A method in accordance with claim 1, characterized in that an optical measuring device (14) is used with at least one camera such as in particular a full frame camera.

9. A method in accordance with claim 1, characterized in that flashlight of such high energy is used that an image having definition in depth is still obtained by means of the camera even at higher felt speeds.

10. A method in accordance with claim 1, characterized in that the optical measuring unit is guided in a traversing manner over the width of the felt.

11. A method in accordance with claim 1, characterized in that the camera and the lighting are used such that they stand at a specific angle to one another.

12. A method in accordance with claim 1, characterized in that sectional felt conditioning measures are initiated in the case of a sectional error recognition.

13. A method in accordance with claim 1, characterized in that the felt conditioning measures initiated in the event of an error recognition include the production of a tube suction vacuum, the selection of spray tube nozzles and/or the like.

14. A device (10) for monitoring the state of a felt (12) serving for the dewatering of a fiber web, in particular of a paper web or of a cardboard web, comprising at least one optical measuring device (14) for the measurement of the felt topography and/or for the production of an image of the felt surface having definition in depth and comprising an evaluation and/or control unit (16) to compare the obtained measured result with previously stored error samples and to initiate corresponding felt conditioning measures, preferably automatically, in dependence on the result of the comparison.

15. A device in accordance with claim 14, characterized in that a plan image and/or an oblique image of the felt surface is produced by means of the optical measuring device (14).

16. A device in accordance with claim 14, characterized in that means (16) are provided to record and/or to store the image produced by means of the optical measuring device.

17. A device in accordance with claim 14, characterized in that means (16) are provided to record and/or to store the image produced by means of the optical measuring device.

18. A device in accordance with claim 14, characterized in that the image produced by means of the optical measuring device (14) is only temporarily stored and is deleted or overwritten again after a comparison has been made with the stored error patterns.

19. A device in accordance with claim 14, characterized in that the felt (12) is monitored continuously or periodically by means of the optical measuring device (14).

20. A device in accordance with claim 14, characterized in that the felt monitoring takes place online.

21. A device in accordance with claim 14, characterized in that an optical measuring device (14) is provided having at least one camera such as in particular a full frame camera.

22. A device in accordance with claim 14, characterized in that means are provided to produce flashlight of such high energy that an image having definition in depth is still obtained by means of the camera in particular also at higher felt speeds.

23. A device in accordance with claim 14, characterized in that the optical measuring unit (14) is guided in a traversing manner over the width of the felt.

24. A device in accordance with claim 14, characterized in that the camera and the lighting stand at a specific angle to one another.

25. A device in accordance with claim 14, characterized in that sectional felt conditioning measures are initiated in the case of a sectional error recognition.

26. A device in accordance with claim 14, characterized in that the felt conditioning measures initiated in the event of an error recognition include the production of a tube suction vacuum, the selection of spray tube nozzles and/or the like.

27. A method of monitoring the state of a felt or screen (22) serving for the dewatering of a fiber web, in particular of a paper web or of a cardboard web, in which specific felt and/or screen properties are determined permanently or cyclically at intervals and are filed under a pre-settable felt and/or screen name in a historical database, and in which, by means of an evaluation and/or control unit (26), the relevant data are graphically recorded, in particular relative to the time of the felt and/or screen change, the obtained property development over time is compared with a felt and/or screen property development already previously determined or recorded and a signaling is initiated and/or at least one counter measure is automatically triggered if the obtained property development over time differs by a pre-settable measure from the felt and/or screen property development over time already previously determined or recorded or lies outside a pre-settable range.

28. A method in accordance with claim 27, characterized in that at least one counter measure is triggered sectionally over the width of the felt and/or of the screen.

29. A method in accordance with claim 27, characterized in that the development of the relevant data over time is recorded.

30. A method in accordance with claim 27, characterized in that the development of the relevant data over time is recorded.

31. A method in accordance with claim 27, characterized in that a processing and/or monitoring and/or controlling of sectional qualities of felt and/or screen properties, a sectional monitoring of the felt and/or screen properties and/or a monitoring or recognition of specific boundaries and error patterns is provided for the selection and initiation of specific counter measures, in each case over time and/or sectionally over the width of the machine.

32. A method in accordance with claim 27, characterized in that a characteristic field control is provided.

33. A method in accordance with claim 27, characterized in that, optionally, at least one of the following counter measures is automatically carried out:
change in spray tube pressures;
change in tube suction vacuums;
activation of at least one further operating parameter which influences at least one monitored felt and/or screen property,
with the relevant values in particular being put into a pre-set characteristic field again and with a corresponding feedback loop preferably being used.

34. A method in accordance with claim 33, characterized in that at least the changes in spray tube pressures and/or the changes in tube suction vacuums take place sectionally.

35. A method in accordance with claim 27, characterized in that the felt and/or screen properties determined and/or recorded are put into relationship with a mean property trend value already previously determined or recorded and/or are compared with this.

36. A method in accordance with claim 27, characterized in that the felt and/or screen properties are determined online.

37. A method in accordance with any one of the claim 24, characterized in that the felt and/or screen properties are determined offline.

38. A method in accordance with any one of the claims 25, characterized in that the felt and/or screen properties are determined offline.

39. A method in accordance with any one of the claims 26, characterized in that the felt and/or screen properties are determined offline.

40. A method in accordance with any one of the claims 27, characterized in that the felt and/or screen properties are determined offline.

41. A method in accordance with any one of the claims 28, characterized in that the felt and/or screen properties are determined offline.

42. A method in accordance with any one of the claims 29, characterized in that the felt and/or screen properties are determined offline.

43. A method in accordance with any one of the claims 30, characterized in that the felt and/or screen properties are determined offline.

44. A method in accordance with any one of the claims 32, characterized in that the felt and/or screen properties are determined offline.

45. A method in accordance with claim 27, characterized in that at least the occurring felt and/or screen vibrations are determined as the felt and/or screen properties.

46. A method in accordance with claim 27, characterized in that at least the felt and/or screen permeabilities are determined as the felt and/or screen properties.

47. A method in accordance with claim 27, characterized in that at least the water content of the felt and/or screen (22) is determined as the felt and/or screen property.

48. A method in accordance with claim 27, characterized in that at least the felt and/or screen contamination is determined as the felt and/or screen property.

49. A method in accordance with claim 27, characterized in that at least the surface finish of the felt and/or screen is determined as the felt and/or screen property.

50. A device (20) for monitoring the state of a felt or screen (22) serving for the dewatering of a fiber web, in particular of a paper web or of a cardboard web, in which specific felt and/or screen properties are determined permanently or cyclically at intervals and are stored under a pre-settable felt and/or screen name in a historical database, comprising an evaluation and/or control unit (26) for the graphical recording of the relevant data, in particular relative to the time of the felt and/or screen change, for the comparison of the obtained property development over time with at least one felt and/or screen property development over time already previously determined or recorded and for the signaling and/or automatic triggering of at least one counter measure in the event of a deviation of the obtained property development over time from the felt and/or screen property development over time already previously determined or recorded by a pre-settable measure or in the event of a property development over time lying outside a pre-settable range.

51. A device in accordance with claim 50, characterized in that at least one counter measure can be triggered sectionally over the felt and/or screen width.

52. A device in accordance with claim 50, characterized in that the development over time of the relevant data is recorded.

53. A device in accordance with claim 51, characterized in that the development over time of the relevant data is recorded.

54. A device in accordance with claim 50, characterized in that means are provided for the processing and/or monitoring and/or controlling of sectional qualities of felt or screen properties, for the sectional monitoring of the felt and/or screen properties and/or for the monitoring or recognition of certain thresholds and error patterns for the selection and initiation of specific counter measures, in each case over time and/or sectionally over the machine width.

55. A device in accordance with claim 50, characterized in that a characteristic field control is provided.

56. A device in accordance with claim 50, characterized in that, optionally, at least one of the following counter measures can be automatically triggered:
change in spray tube pressures
change in tube suction vacuums
activation of at least one further operating parameter which influences at least one monitored felt and/or screen property,
with the relevant values in particular again being put into a pre-set characteristic field and with a corresponding feedback loop preferably being used.

57. A device in accordance with claim 52, characterized in that at least the spray tube pressures and/or the tube suction vacuums can be sectionally changed.

58. A device in accordance with claim 50, characterized in that the determined and/or recorded felt and/or screen properties are put into relationship with a mean property trend value already previously determined or recorded and/or are compared with this in the evaluation and/or control unit.

59. A device in accordance with claim 50, characterized in that the felt and/or screen properties are determined online.

60. A device in accordance with any one of claims 50, characterized in that the felt and/or screen properties are determined offline.

61. A device in accordance with any one of claims 50, characterized in that the felt and/or screen properties are determined offline.

62. A device in accordance with any one of claims 51, characterized in that the felt and/or screen properties are determined offline.

63. A device in accordance with any one of claims 52, characterized in that the felt and/or screen properties are determined offline.

64. A device in accordance with any one of claims 53, characterized in that the felt and/or screen properties are determined offline.

65. A device in accordance with any one of claims 54, characterized in that the felt and/or screen properties are determined offline.

66. A device in accordance with any one of claims 55, characterized in that the felt and/or screen properties are determined offline.

67. A device in accordance with any one of claims 56, characterized in that the felt and/or screen properties are determined offline.

68. A device in accordance with any one of claims 57, characterized in that the felt and/or screen properties are determined offline.

69. A device in accordance with claim 50, characterized in that at least the occurring felt and/or screen vibrations are determined as the felt and/or screen properties.

70. A device in accordance with claim 50, characterized in that at least the felt and/or screen permeabilities are determined as the felt and/or screen properties.

71. A device in accordance with claim 50, characterized in that at least the water content of the felt and/or screen (22) is determined as the felt and/or screen property.

72. A device in accordance with claim 50, characterized in that at least the felt and/or screen contamination is determined as the felt and/or screen property.

73. A device in accordance with claim 50, characterized in that at least the surface finish of the felt and/or screen is determined as the felt and/or screen property.
